(19) United States
(12) Patent Application Publication
(10) Pub. No.: US 2024/0050244 A1
(43) Pub. Date: Feb. 15, 2024

(54) Advanced Rotation Restrictor

(71) Applicant: Howmedica Osteonics Corp., Mahwah, NJ (US)

(72) Inventors: Shashank Verma, Agra (IN); Rajan Yadav, New Delhi (IN); Roy Philip Splieth, Central Valley, NY (US); Travis Geels, Fort Wayne, IN (US); Matt Kartholl, Fort Wayne, IN (US); Sunny Shorabh, Ghaziabad (IN); Purvi Bhatia, Faridabad (IN)

(73) Assignee: Howmedica Osteonics Corp., Mahwah, NJ (US)

(21) Appl. No.: 18/230,207

(22) Filed: Aug. 4, 2023

Related U.S. Application Data
(60) Provisional application No. 63/397,986, filed on Aug. 15, 2022.

(51) Int. Cl. A61F 2/46; A61F 2/40

(52) U.S. Cl. CPC A61F 2/4684; A61F 2/4059; A61F 2002/30471

(57) ABSTRACT

A prosthetic trial base includes a hinge and two levers connected to one another by the hinge. Each lever includes a tab extending proximally of the hinge and a fin extending distally of the hinge. The prosthetic trial base also includes a head movable between the tabs to force the tabs apart from one another. The trial base also includes a collar defining a bore in which the tabs are received and a lid that is connected to the levers. The head and is connected to the lid in a manner that enables the head to be driven relative to the lid.

### CROSS-REFERENCE TO RELATED APPLICATIONS

This application claims the benefit of the filing date of U.S. Provisional Patent Application No. 63/397,986 filed Aug. 15, 2022, the disclosure of which is hereby incorporated herein by reference in its entirety.

### BACKGROUND

A joint replacement procedure is sometimes necessary to repair a joint having a diseased or damaged articulating surface. Such a procedure involves removal of the diseased or damaged portions of the joint and replacing them with a prosthetic implant. This is often a desirable procedure for ball-and-socket type joints, particularly the shoulder and hip joints. A shoulder joint replacement procedure, for example, often involves removal of the humeral head and replacement thereof with an implant including a stem and a head. It is important that the implant be positioned correctly within the joint in order to ensure that appropriate joint kinematics, including range of motion, are preserved so as to replicate, as closely as possible, those of the original joint.

The structure of prosthetic joint components has been developed to be suited for permanent implantation into the joint and includes features that may promote bony ingrowth, adhesion using cement, press-fit or a combination thereof. Particularly, in the case of implants including a stem, such as those used in shoulder arthroplasty, these features are included on the outside surface of the stem. Such features are not well-suited for use during the assessment of joint kinematics. Accordingly, instruments to be used in this part of the procedure have been developed. For shoulder arthroplasty, two general types of such devices have been developed, both of which are affixed to the bone during joint kinematic evaluation and removed therefrom after a proper position for the implant has been determined.

One such device is an externally-fixated jig. These devices, however tend to be bulky, complex and time consuming to set up and use. They also require the use of external fixation devices and/or power tools that introduce further complications to the surgical procedure. Because these devices exit through the incision to the outside of the body they are generally cumbersome making accurate range of motion assessment difficult. Such jigs are shown in U.S. Pat. Nos. 6,267,785 and 6,193,758.

An additional or alternative device is a trial. Typically, these devices are designed to correspond to an implant in size and shape. The trial is designed to be temporarily inserted into a prepared medullary canal of the humerus in a manner similar to that of an implant. Known trials are typically used in conjunction with a lap sponge that is temporarily wrapped around the distal portion of the trial. The sponge-wrapped trial is then wedged into the canal, the sponge promoting a pressure fit therebetween, to evaluate the appropriate position for the implant. In the alternative, a foam ring can be assembled onto the trial stem as discussed in the brochure entitled Zimmer Trabecular Metal™ Humeral Stem Four-Part Fracture Surgical Technique, available from Zimmer, Inc., P.O. Box 708, 1800 West Center Street, Warsaw, In 46581-0708. While eliminating the problems associated with externally-fixated devices, known trials present other problems. For example, by positioning a resilient material, such as a sponge, between the inside surface of the prepared medullary canal and the trial stem, the stem may be allowed to move within the joint leading to an unreliable joint kinematics assessment. Additionally, the use of a lap sponge to hold the trial within the medullary canal leads to a risk of leaving sponge or cloth debris behind in the bone after the trial is removed. Therefore, further improvements are desirable.

### BRIEF SUMMARY

According to some aspects, a base for a trial prosthesis, or a trial base, may include a stem for insertion into a medullary canal and features for engaging the bone to inhibit rotation of the stem relative to the bone within the medullary canal. Such features may be fins that define at least a portion of the stem in addition to a mechanism for driving the fins apart. The fins may be hingedly or flexibly connected to one another, and the mechanism may be configured to cause the fins to move relative to the hinged or flexible connection. In some arrangements, the fins may each be a part of a lever, and each lever may include a tab on an opposite side of the hinge from the fin. In such arrangements, the mechanism may include a collar surrounding the tabs and a bolt drivable relative to the collar and into a space between the tabs to act as a wedge and force the tabs apart. In other arrangements, the mechanism may include a cam and a rod extending between the fins, with the cam either being fixed to the rod between the fins so that rotating the rod causes the cam to force the fins apart or being positioned at a proximal end of the rod so that rotating the cam can force the rod to move axially to act as a wedge and force the fins apart.

In other arrangements, the features for engaging the bone to inhibit rotation of the stem relative to the bone may be provided by an anchor fastenable to the stem, or to another portion of the trial base that is connected to the stem. The anchor may include an extension so that a portion of a bone may be clamped between the extension and the stem when the anchor is fastened to the stem or other portion of the trial base that is connected to the stem. In other arrangements, the features for engaging the bone to inhibit rotation of the stem relative to the bone may be provided by one or more tabs extending along and outside the bone, so that some portion of the bone may be between the stem and the one or more tabs. In such arrangements, the tabs may include holes for accepting fasteners, such as set screws, that may be driven through the tabs and into the bone.

### BRIEF DESCRIPTION OF THE DRAWINGS

FIG. 1A is an elevation view of a stem of a trial base according to an arrangement.

FIG. 1B is an oblique perspective view of the stem of a trial base of FIG. 1A with a collar thereon.

FIG. 1C is an oblique perspective view of a lid.

FIG. 1D is an oblique perspective view of a trial base including the stem of FIG. 1A, the collar of FIG. 1B, and the lid of FIG. 1C.

FIG. 1E is a cross-section of the trial base of FIG. 1D in an unexpanded state.

FIG. 1F is a cross-sectional view of the trial base of FIG. 1E in an expanded state.

FIG. 2A is a partial transparent view of a trial base according to another arrangement.

FIG. 2B is a cross-section of a proximal portion of a trial base according to another arrangement.

FIG. 3A is an oblique perspective view of a proximal portion of a trial base according to another arrangement.

FIG. 3B is an oblique perspective view of an anchor of the trial base of FIG. 3A.

FIG. 3C is an elevation view of the trial base of FIG. 3A.

FIG. 4A is an elevation view of the proximal portion of a trial base according to another arrangement.

FIG. 4B is a cross-section of the proximal portion of a trial base of FIG. 4A.

FIG. 5A is an oblique perspective view of a proximal portion of a trial base according to another arrangement.

FIG. 5B is an oblique perspective view of an assembly including the proximal portion of a trial base of FIG. 5A and a clip.

FIG. 5C is an oblique perspective view of an assembly including the assembly of FIG. 5B and an anchor.

FIG. 5D is an oblique perspective view of the clip and the anchor of FIGS. 5B and 5C in an exploded state.

FIG. 5E is an oblique perspective view of a proximal side of the pin and the anchor of FIGS. 5B-5D in an assembled state.

FIG. 5F is an oblique perspective view of a distal side of the clip and the anchor of FIGS. 5B-5E in an assembled state.

FIG. 6A is an elevation view of a trial base according to another arrangement.

FIG. 6B is an elevation view of a cam rod of the trial base of FIG. 6A.

FIG. 6C is an elevation view of a distal end of the trial base of FIG. 6A in an unexpanded state.

FIG. 6D is an elevation view of the distal end of the trial base of FIG. 6A in an expanded state.

FIG. 7A is a cross-section of a trial base according to another arrangement.

FIG. 7B is a cross-section of the trial base of FIG. 7A on a plane that is orthogonal to the plane of FIG. 7A.

FIG. 8 is an oblique perspective view of a trial base according to another arrangement.

### DETAILED DESCRIPTION

As used herein, the term “proximal,” when used in connection with a surgical tool or device, or components of a device, refers to the end of the device closer to the user of the device when the device is being used as intended. On the other hand, the term “distal,” when used in connection with a surgical tool or device, or components of a device, refers to the end of the device farther away from the user when the device is being used as intended. As used herein, the terms “substantially,” “generally,” “approximately,” and “about” are intended to mean that slight deviations from absolute are included within the scope of the term so modified, such as deviations of up to 10% greater or lesser than absolute. All vertical directional terms, such as “up,” “down,” “above,” “below,” “vertical,” or “height” used in the following description refer only to the orientation of features as depicted in the figure being described. Such directional terms are not intended to suggest that any features of the devices described herein must exist in any particular orientation when constructed.

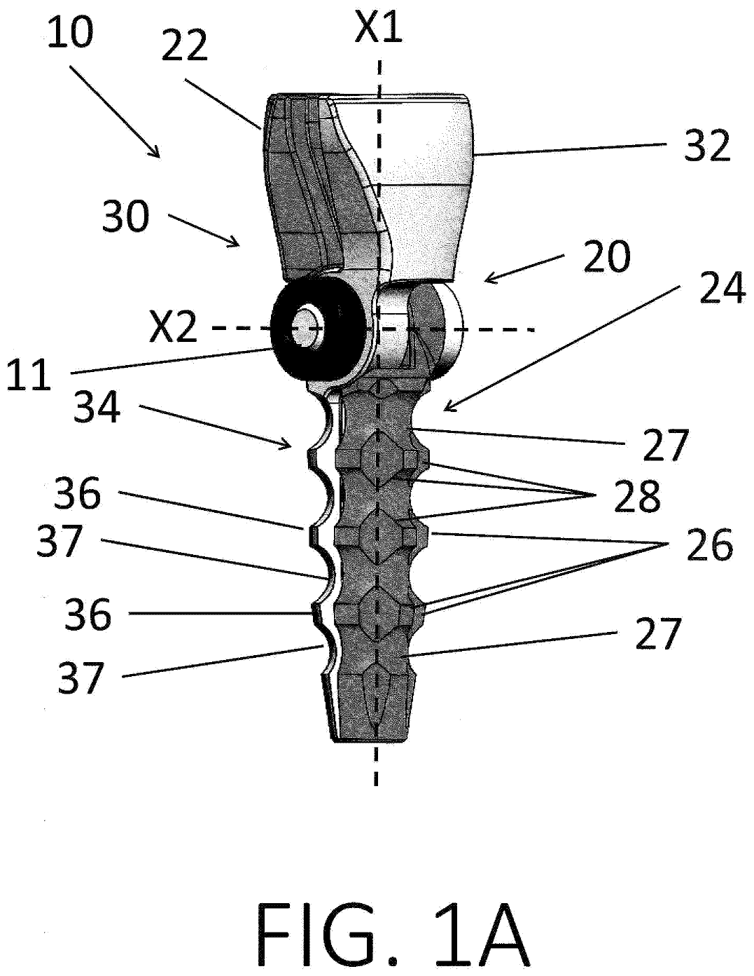

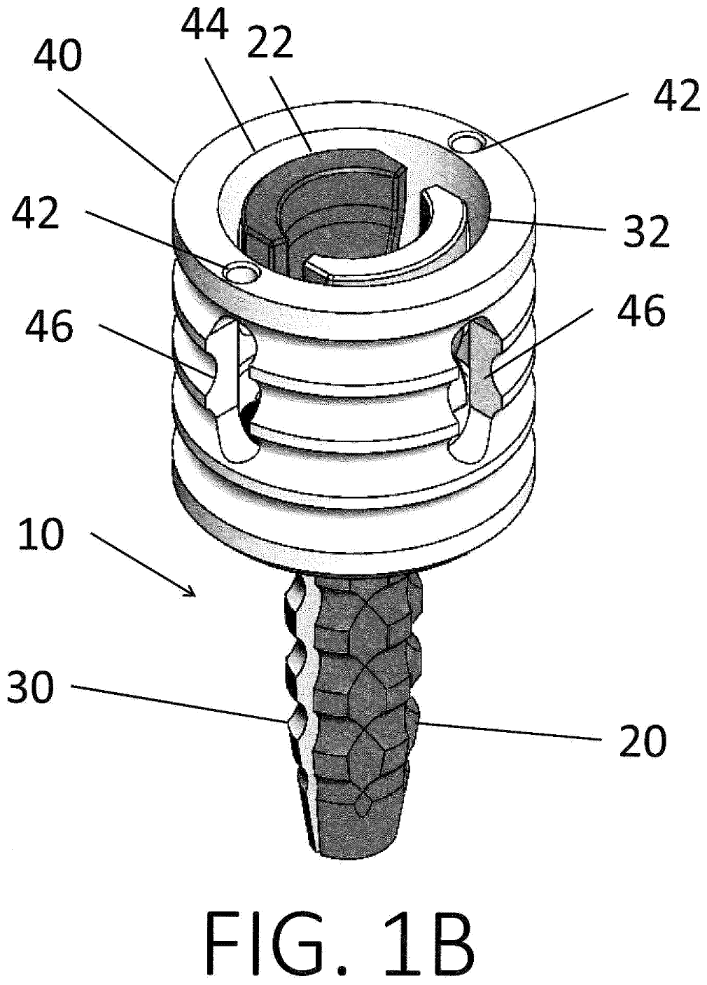

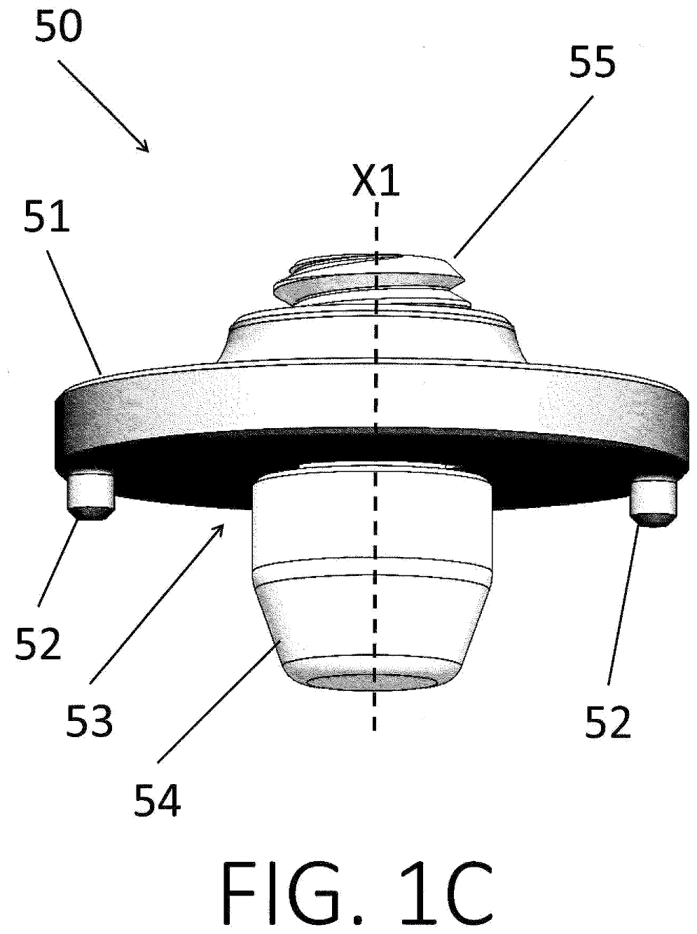

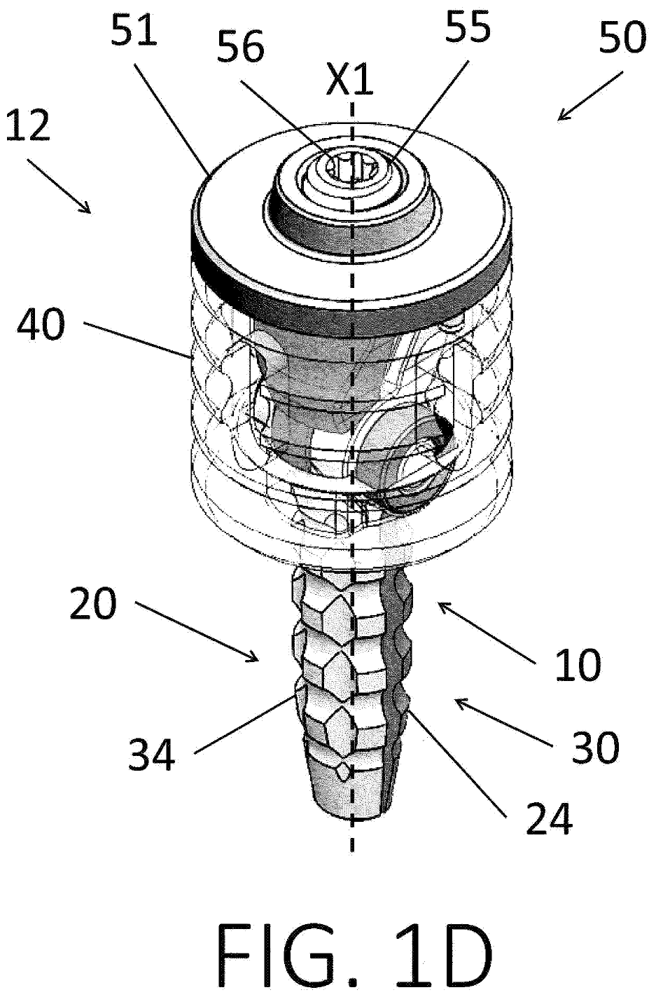

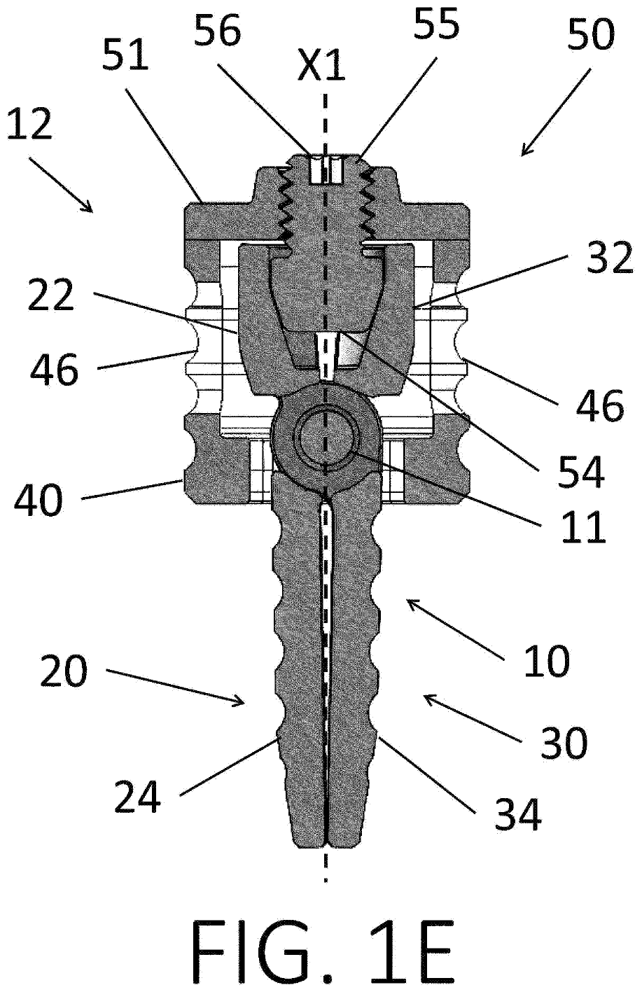

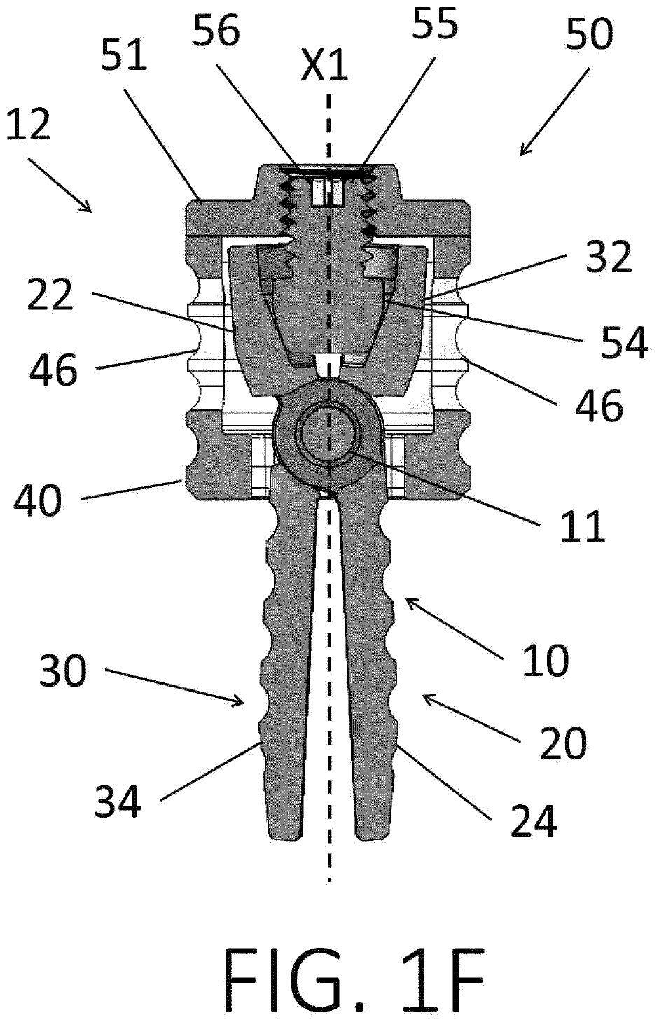

FIG. 1A illustrates a stem 10 of a trial base 12 that is shown in FIG. 1D. As used herein, a trial base refers to a portion of a trial to which an articular portion of a trial prosthesis is or may be attached. The examples in the present disclosure are described as being trial bases for trial humeral prostheses. Thus, an articular portion in the form of a trial humeral head, not illustrated, may be mounted to a proximal end of any of the trial bases of the present disclosure. However, in other examples, the trial humeral head could be integrally formed on a proximal end of trial bases otherwise according to any of the arrangements disclosed herein. In still further examples, the trial bases described herein could be implemented with mountable or integrally formed articular portions for trial prostheses for replacing any other bone.

Stem 10 includes a first lever 20, a second lever 30, and a hinge 11 that joins first lever 20 to second lever 30. Hinge 11 of the present example is specifically a non-living hinge. First lever 20 includes a tab 22 proximal of hinge 11 and fin 24 distal of hinge 11. Similarly, second lever 30 includes a tab 32 proximal of hinge 11 and a fin 34 distal of hinge 11. Hinge 11 connects levers 20, 30 to one another such that forcing tabs 22, 32 away from one another also forces fins 24, 34 away from one another. Thus, an angle between fins 24, 34 will increase as an angle between tabs 22, 32 increases, meaning the angle between fins 24, 34 and the angle between tabs 22, 32 is directly correlated. Fins 24, 34 include respective protrusions 26, 36 that extend radially away from longitudinal axis X1 for engaging a bone from within the bone's medullary canal. Protrusions 26, 36 are defined between circumferential grooves 27, 37 and lateral grooves 28, 38 extending on radially outer surfaces of the respective fins 24, 34. In other arrangements, protrusions 26, 36 may be shaped or defined differently, replaced by different features for engaging bone, or omitted altogether.

Stem 10 extends from a proximal end to a distal end along a longitudinal axis X1 and hinge 11 enables levers 20, 30 to rotate relative to one another about a lateral axis X2. In each arrangement within the present disclosure, proximal and distal are used with respect to an implanted position of the trial within the medullary canal of a humerus and wherein longitudinal axis X1 is at least approximately aligned on the axis of the humerus. However, this use of the terms proximal and distal is not intended to suggest that the trials of the present disclosure would be unsuitable for implanting in other orientations. In the illustrated arrangements, lateral axis X2 is orthogonal to longitudinal axis X1 and intersects longitudinal axis X1, though in other examples lateral axis X2 may be offset from longitudinal axis X1.

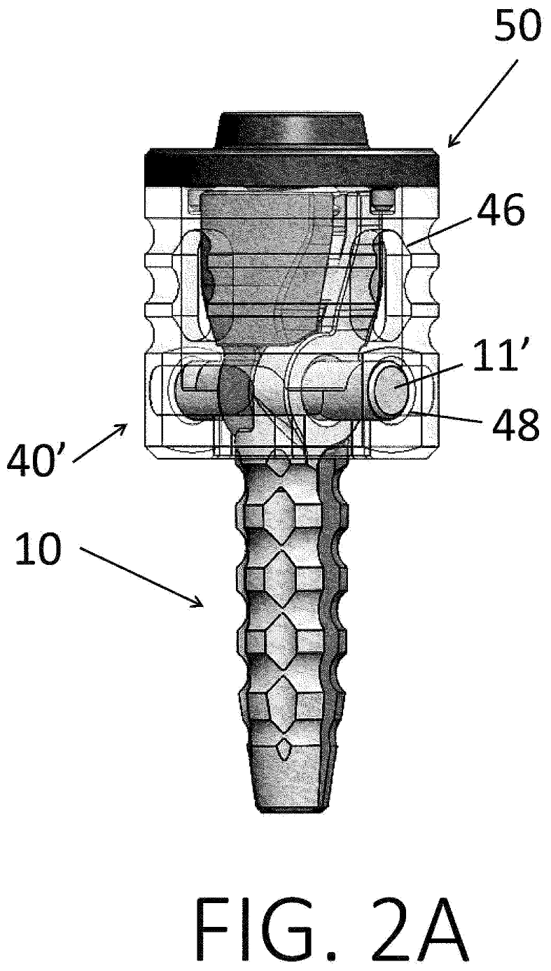

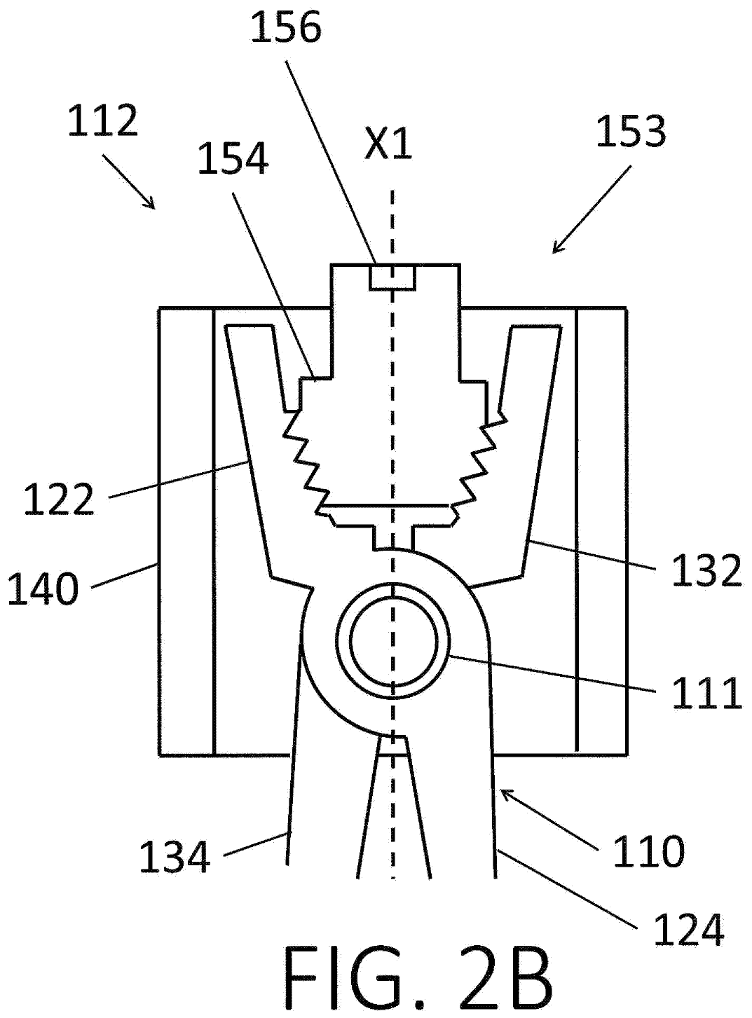

FIG. 1B shows stem 10 with a collar 40 assembled thereon. Collar 40 is connected to stem 10 in a manner that prevents collar 40 from rotating about or travelling along longitudinal axis X1, particularly during operation of a bolt 53 of a lid 50, described in more detail below. For example, hinge 11 may be captured by an interior of collar 40, such as a recess or indentation in an inner surface of collar 40, that prevents collar 40 from moving axially relative to stem 10. In an alternative embodiment, such as that shown in FIG. 2B, a hinge 11′ may extend through a through-hole in collar 40 so that hinge 11′ can be assembled from an exterior of collar 40 and secured to collar 40. In such embodiment, hinge 11′ may be welded to collar 40 or otherwise secured thereto, such as via a press-fit or snap-fit arrangement, which may help provide additional stability to hinge 11′ relative to that of hinge 11. Fins 22, 24 are located within a bore 44 of collar 40 and can therefore be accessed from a proximal end of collar 40. Holes 42 are defined in a proximal surface of collar 40 adjacent bore 44. Collar 40 may optionally include slots 46 on lateral sides thereof, as shown in the illustrated example, to facilitate cleaning. Collar 40 may limit a possible depth of insertion of trial base 12 by sitting atop a prepared surface of a bone into which trial base 12 is inserted.

FIG. 1C illustrates a lid 50 that includes a disk 51 and a bolt 53. Bolt 53 includes a threaded shank 55 extending along longitudinal axis X1 and engaged with a threaded hole defined through disk 51 so that turning bolt 53 about longitudinal axis X1 relative to disk 51 will cause bolt 53 to advance either proximally or distally along longitudinal axis X1 relative to disk 51. Head 54 defines a distal end of bolt 53, so advancing bolt 53 along longitudinal axis X1 by rotating bolt 53 relative to disk 51 will also cause head 54 to move proximally or distally along longitudinal axis X1.

Lid 50 also includes pegs 52 extending from a distal side of disk 51. Pegs 52 match holes 42 in size and location without exceeding the quantity of holes 42. Lid 50 may therefore be installed on a proximal end of collar 40 to form trial base 12 as shown in FIG. 1D so that pegs 52 are received in holes 42 to restrict rotation of disk 51 relative to collar 40 about longitudinal axis X1 and to facilitate assembly between collar 40 and lid 50. In other arrangements, pegs may additionally or instead be provided on a proximal side of collar 40 while corresponding holes are provided on a distal side of disk 51. In still further arrangements, collar 40 and disk 51 may additionally or instead be provided with other features for limiting rotation of disk 51 about longitudinal axis X1 relative to collar 40. Lid 50 may be secured to collar 40 to prevent lid 50 from being lifted therefrom during operation of bolt 53. In this regard, lid 50 may be welded to collar 40 or secured in some other manner, such as via a press-fit, snap-fit, or threaded engagement, for example.

Because rotation of disk 51 about longitudinal axis X1 relative to collar 40 is restricted when trial base 12 is assembled as shown in FIG. 1D, rotating bolt 53 about central axis X1 will cause head 54 to travel proximally or distally between tabs 22, 32 within collar 40. A proximal end of bolt 53 may optionally include an adaptor 56, such as the polygonal socket of the illustrated example, for connecting a rotational driving tool to bolt 53. Adaptor may be any convex or concave feature enabling transmission of torque about longitudinal axis X1 to bolt 53.

As shown in FIGS. 1E and 1F, tabs 22, 32 and head 54 are respectively configured so that driving head 54 distally relative to tabs 22, 32 forces tabs 22, 32 apart from one another and radially away from central axis X1. Thus, trial base 12 may be used by first preparing a humerus, or other bone, to accept a prosthesis, including clearing space in medullary canal to accept stem 10 if necessary, inserting fins 24, 34 into the prepared medullary canal, then driving bolt 53 distally within collar 40 to force fins 24, 34 radially outward until fins 24, 34 engage the prepared bone from within. Trial base 12 should be placed at an intended depth and angular position before bolt 53 is driven distally because the engagement of the bone by fins 24, 34 will inhibit rotation of trial base 12 about longitudinal axis X1 and travel of trial base 12 along longitudinal axis X1 relative to the engaged bone. An articular portion of a trial prosthesis, such as a trial prosthetic humeral head, may be attached to trial base 12 at any time during use of trial base 12. The articular portion may optionally be attached after fins 22, 32 are inserted into the medullary canal so that trial base 12 is not made heavy or otherwise difficult to handle during insertion. The articular portion may optionally be attached after bolt 53 is driven to cause fins 22, 32 to engage the bone so that the proximal end of bolt 53 remains unobstructed until the driving is completed. While fins 22, 32 engage the bone, a trialing process may be conducted by attaching and removing multiple different trial articular portions to trial base 12 in series to assess what size and shape of implant would be most suitable for the patient's anatomy.

In the illustrated example, tabs 22, 32 and head 54 are respectively configured so that driving head 54 distally relative to tabs 22, 32 forces tabs 22, 32 apart because head 54 tapers from being relatively narrow at its distal end and relatively wide at its proximal end to give head 54 a wedge shape. Meanwhile, radially inner surfaces of tabs 22, 32 are in the shape of ramps that are further from longitudinal axis X1 at their proximal ends and nearer to longitudinal axis X2 at their distal ends. However, in other arrangements, tabs 22, 32 and head 54 could be respectively configured in any other way that would cause driving head 54 distally relative to tabs 22, 32 to force tabs 22, 32 apart. For example, head 54 may be cone or dome shaped without radially inner surfaces of tabs 22, 32 being ramped or radially inner surfaces of tabs 22, 32 may be ramped without head 54 being cone or dome shaped.

Though the illustrated arrangement of trial base 12 is configured so that forcing tabs 22, 32 away from one another also forces fins 24, 34 away from one another, arrangements wherein levers 20, 30 are configured so that pulling proximal ends thereof together forces distal ends thereof apart are also contemplated. Additionally, though the illustrated arrangement of trial base is configured so that pushing head 54 of bolt 53 distally forces fins 24, 34 apart, arrangements wherein pushing head 54 distally forces fins 24, 34 apart are also contemplated. Further, though the illustrated arrangement of trial base 12 includes only two levers 20, 30 and two fins 24, 34, stems according to other arrangements may have any plural number of levers and fins.

FIG. 2B illustrates a portion of a trial base 112 according to another arrangement. Trial base 112 is generally alike to trial base 12 in all respects except for those specifically stated or illustrated. Trial base 112 thus includes opposed levers connected at a hinge 111 so that forcing tabs 122, 132, which are located proximally of hinge 111, apart will also drive apart fins 124, 134, which are located distally of hinge 111. Trial base 112 also includes a collar 140 that may sit on a prepared surface of a bone to limit a possible depth of insertion of trial base 112 into the bone.

Though trial base 112 may optionally lack a lid similar to lid 50 described above, trial base 112 includes a bolt 153. Bolt 153 may optionally be provided with an adaptor 156 for a drive tool. Adaptor 156 may have any of the structures described above with respect to adaptor 56. Bolt 153 also includes a threaded head 154 that is in threaded engagement with threads of tabs 122, 132. Thus, rotating bolt 153 about longitudinal axis X1 threadedly advances head 154 between tabs 122, 132. Driving head 154 distally forces tabs 122, 132 apart and thereby causes fins 124, 134 to move away from one another and, if trial base 112 is disposed inside a bone, engage the bone from within.

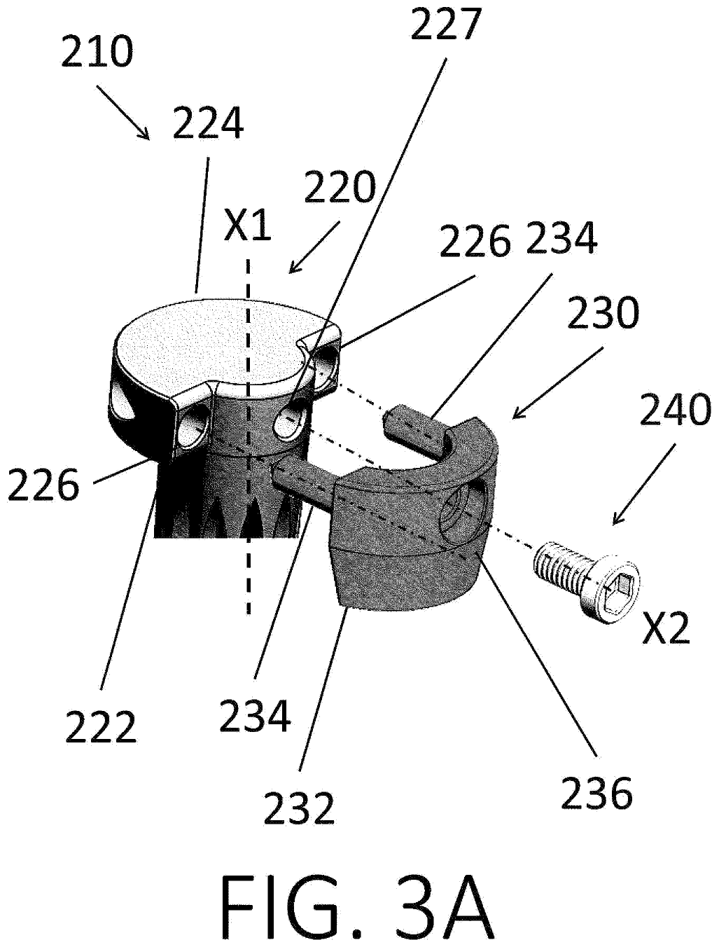

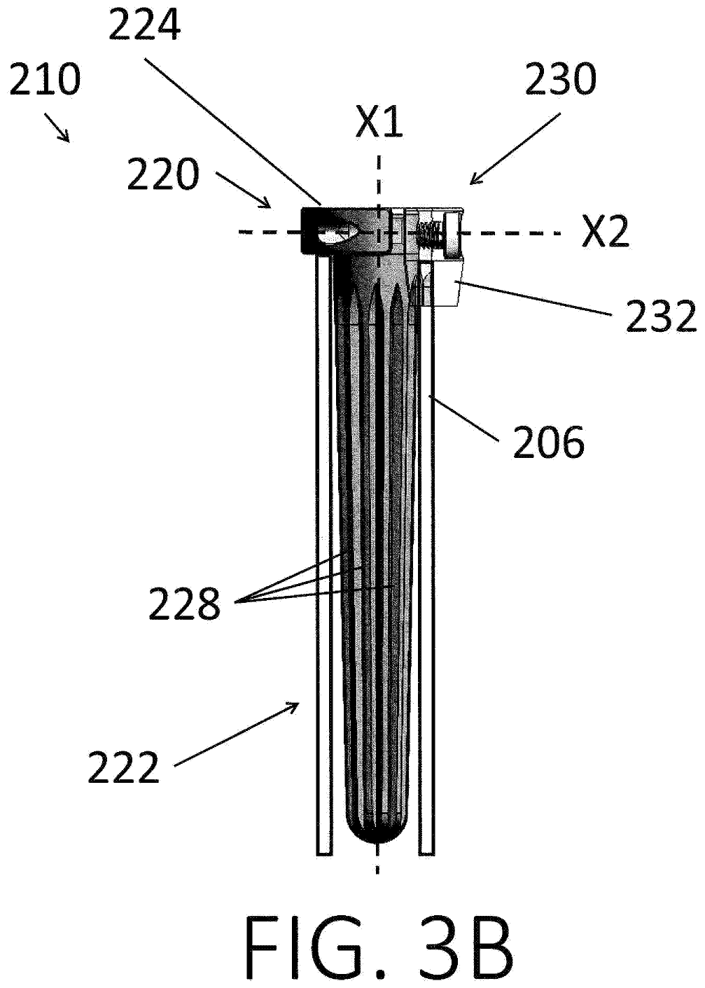

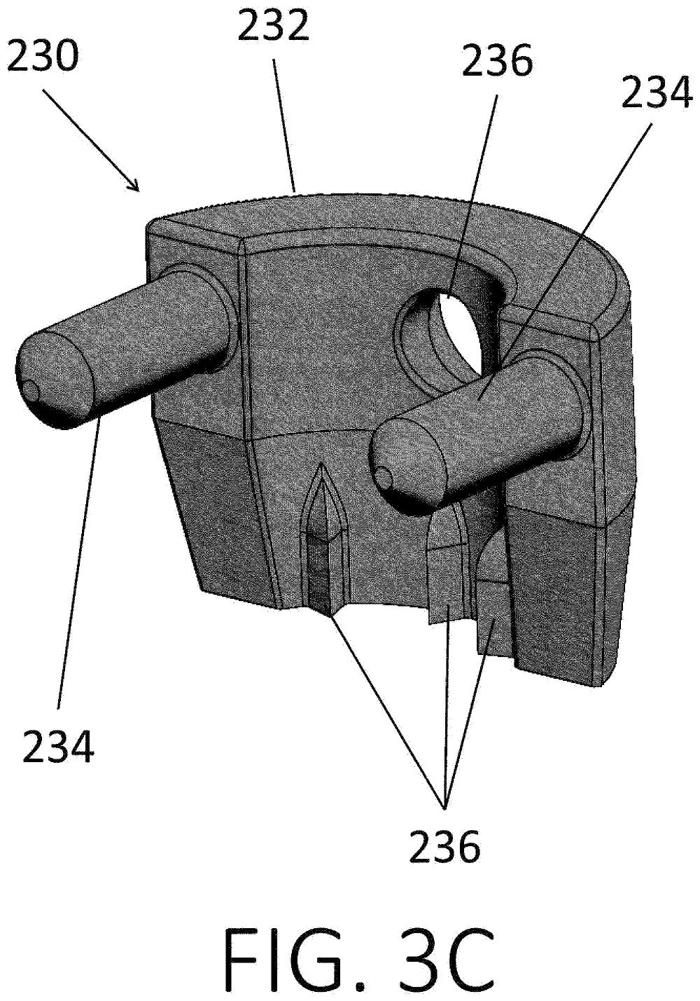

FIGS. 3A-3C illustrates a proximal portion of a trial base 210 that includes a stem 222 and a head 224 at a proximal end of stem 222. Stem 222 is for insertion into the medullary canal of a bone, while head 224 limits the possible depth of insertion by having a greater radius, measured perpendicularly from longitudinal axis X1, than stem 222 in at least one angular direction. Stem 222 may optionally include protrusions, such as the blades 228 that extend radially outward therefrom and longitudinally therealong or the protrusions 26, 36 described above, to resist rotation of stem 222 within the medullary canal. In the illustrated example, head 224 has a greater radius than stem 222 within a range of 180° about central axis X1, though in various other examples the angular range across which head 224 has a greater radius than stem 222 can vary. Head includes holes 226, 227 that extend parallel to lateral axis X2 and all have openings accessible from a same direction that is parallel to lateral axis X2.

Trial base 210 also includes an anchor 230. Anchor 230 includes pegs 234 that are sized and spaced to match holes 226 of head 224. Anchor 230 may therefore be connected to head 224 by translating anchor 230 along lateral axis X2 until pegs 234 are received in holes 226. Anchor 230 also includes an aperture 236 aligned with hole 227 and hole 227 is threaded. Thus, a fastener 240, such as a bolt or screw, may be driven through aperture 236 and threaded into hole 227 to fasten anchor 230 to head 224.

As shown in FIG. 3B, a portion of a bone 206 may be clamped between a distal extension of anchor 230 and stem 222 to inhibit movement of stem 222 relative to bone 206. Particularly, clamping bone 206 between anchor 230 and stem 222 may inhibit rotation of stem 222 relative to bone 206 about longitudinal axis X1_ The alignment of aperture 236 and hole 227 parallel to pegs 234, and thus parallel to the axis along which anchor 230 approaches head 224, enables the clamping of bone 206 to be tightened by threadedly advancing fastener 240 further into hole 227. However, in other examples, similar tightening could be achieved with an anchor 230 designed to approach head 224 along an arc. In yet other examples, aperture 236 and hole 227 may not be parallel to lateral axis X2.

As shown in FIG. 3C, a radially inward side of anchor 230 may optionally include protrusions, such as the blades 236 that extend radially inward therefrom and longitudinally therealong or the protrusions 26, 36 described above, to resist rotation of bone 206 relative to anchor 230 about longitudinal axis X1.

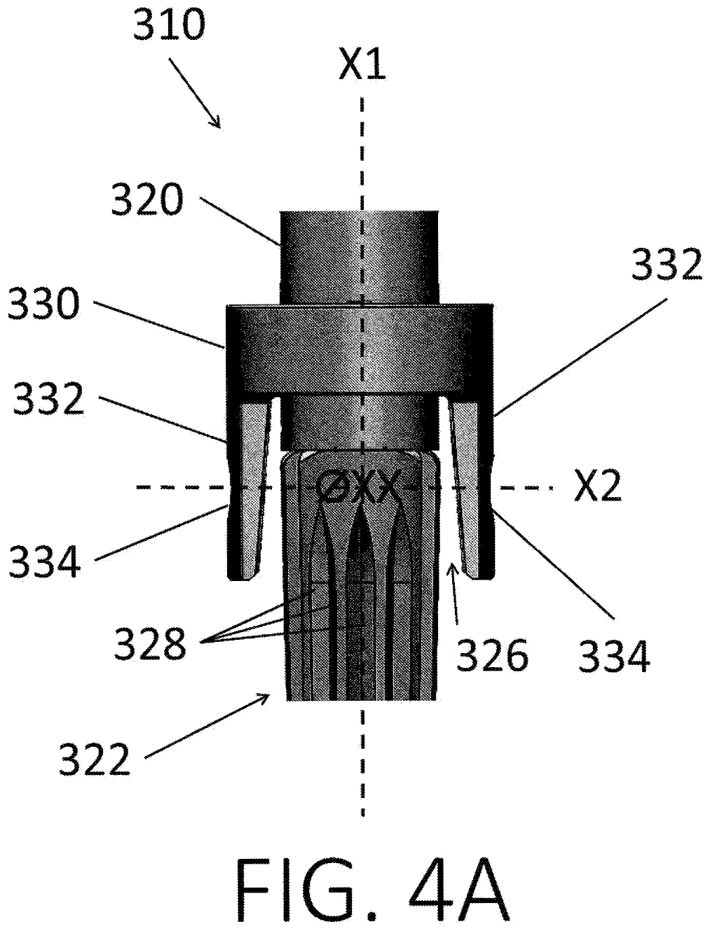

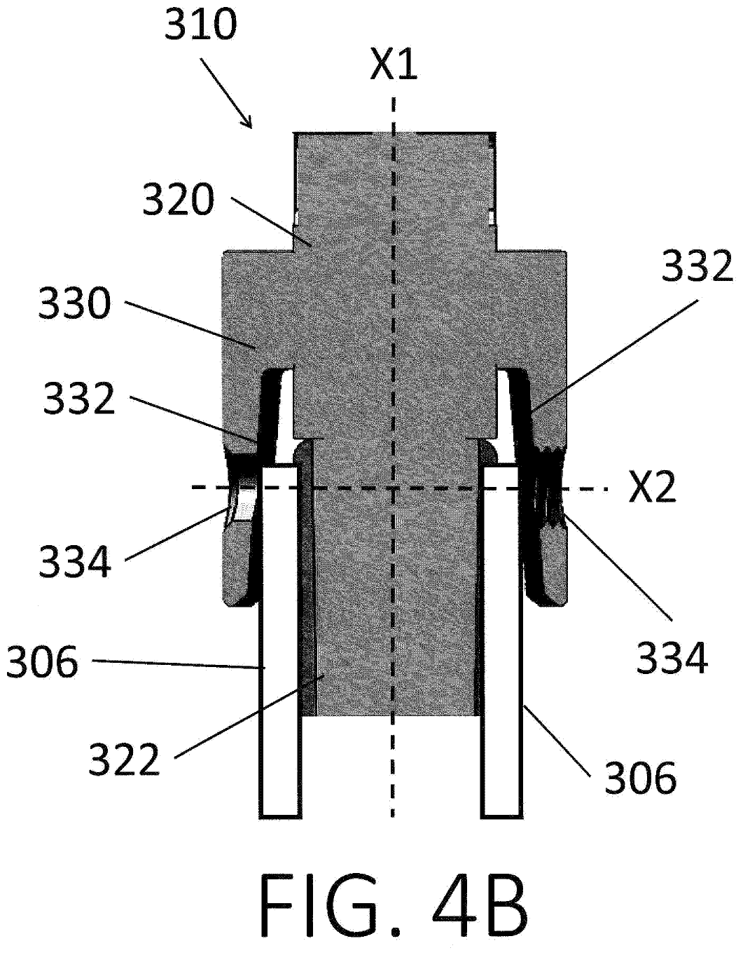

FIGS. 4A and 4B illustrate a proximal portion of a trial base 310 that includes a stem 322 and a collar 330 at a proximal end of stem 322. Stem 322 is for insertion into the medullary canal of a bone, while collar 330 may limit the possible depth of insertion by having a greater radius, measured perpendicularly from longitudinal axis X1, than stem 322 in at least one angular direction. Stem 322 may optionally include protrusions, such as the blades or splines 328 that extend radially outward therefrom and longitudinally therealong or the protrusions 26, 36 described above, to resist rotation of stem 322 within the medullary canal. Trial base 310 may include an adaptor 320 located proximally of stem 322. Collar 330 may optionally be either permanently fixed to adaptor 320 or selectably fixable to adaptor 320 so that collar 330 may be longitudinally slidable along adaptor 320 when unfixed. In the embodiment depicted, adaptor 320 and collar 330 are integrally formed with stem 322 such that they form a monolithic structure. However, as mentioned above, adaptor 320 and/or collar 330 may be modular so that they can be separately connected to stem 322. Adaptor 320 may be configured to enable connection of a trial humeral head prosthesis or a trial of any other kind of prosthesis to trial base 310. In other arrangements, adaptor 320 may be absent and a trial humeral head prosthesis or a trial of any other kind of prosthesis may be integrally formed with a body located where adaptor 320 is located in the illustrated example.

Tabs 332 extend distally from collar 330. Though two tabs 332 are shown in the illustrated example, other arrangements may include one tab 332 or any plural number of tabs 332. Tabs 332 are spaced radially outward from stem 322 so that a radial gap 326 exists between stem 322 and each tab 332. Such gap 326 may taper inwardly, as shown in FIG. 4A, so that gap 326 becomes narrower in a direction toward adaptor 320 or may have a uniform dimension along its extension in a proximal-distal direction. Each tab 332 includes a fastener hole 334. Thus, after stem 322 is inserted into a medullary canal to such depth that bone 306 in which the canal is defined extends to the longitudinal location of holes 334, fasteners may be driven radially inward through holes 334 into the bone 306 extending between stem 322 and tab 332 to fix trial base 310 relative to bone 306, thereby inhibiting at least rotation of trial base 310 about longitudinal axis X1 relative to bone 306. Holes 334 may optionally be threaded or unthreaded, and the fasteners to be driven through holes 334 may be of an unthreaded type, such as a nail, or a threaded type, such as a bolt or screw, accordingly. Though the illustrated arrangement shows two tabs 332 each having a hole 334 aligned on lateral axis X4, individual holes 334 in other examples may be aligned on any other axes that enable driving a fastener into bone 306 through a hole 334. Thus, holes 334 need not be aligned on a common axis as shown in the illustrated example. In addition to a fastener being used in conjunction with holes 334 to help secure collar 330 to bone 306, the narrowing taper of gap 326 may help to grip the end of the bone 306 to further stabilize trial base 310 during a trialing phase of a surgical procedure.

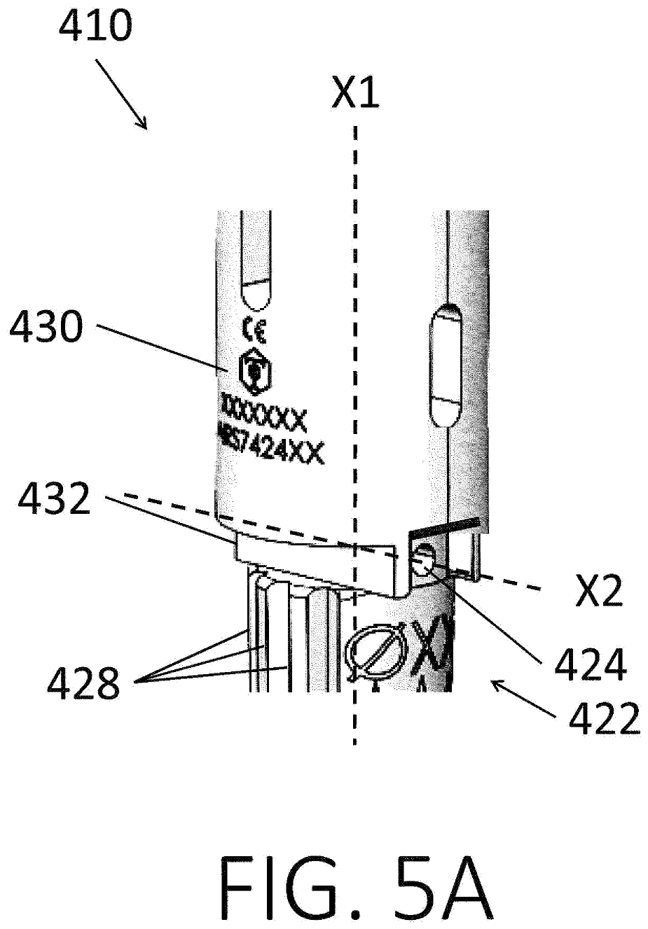

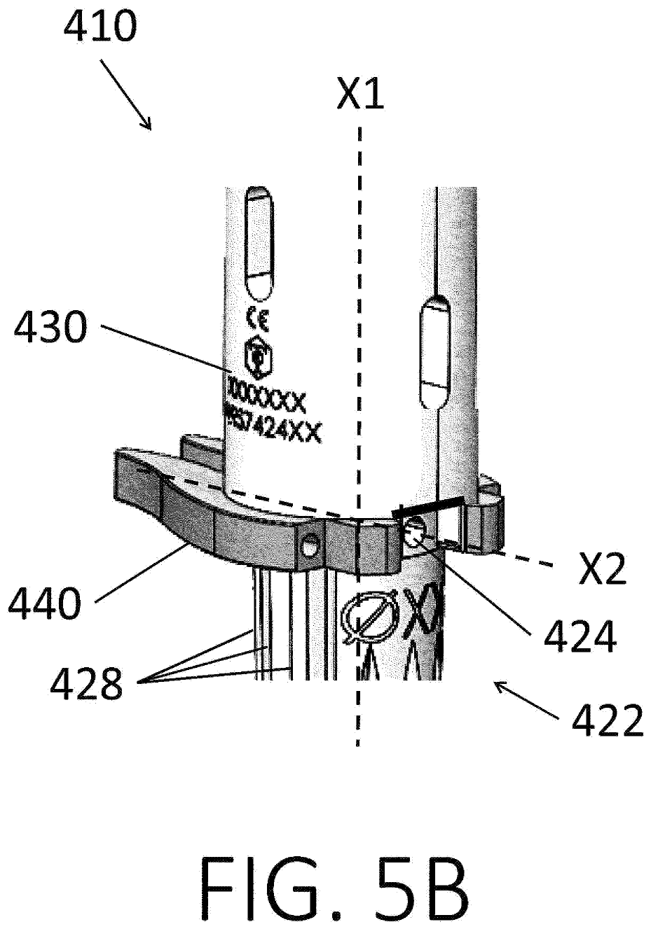

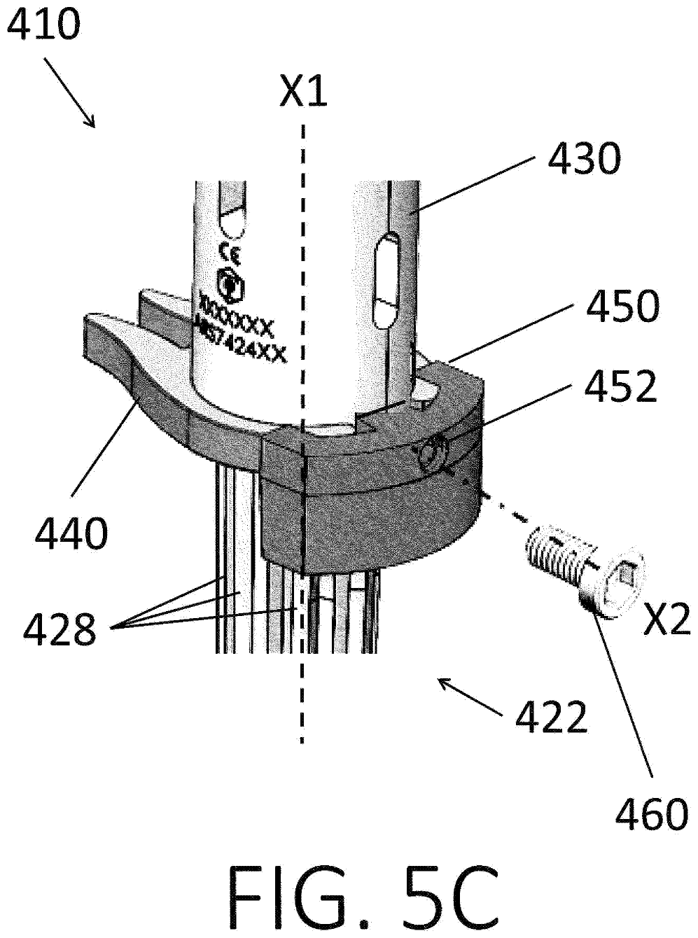

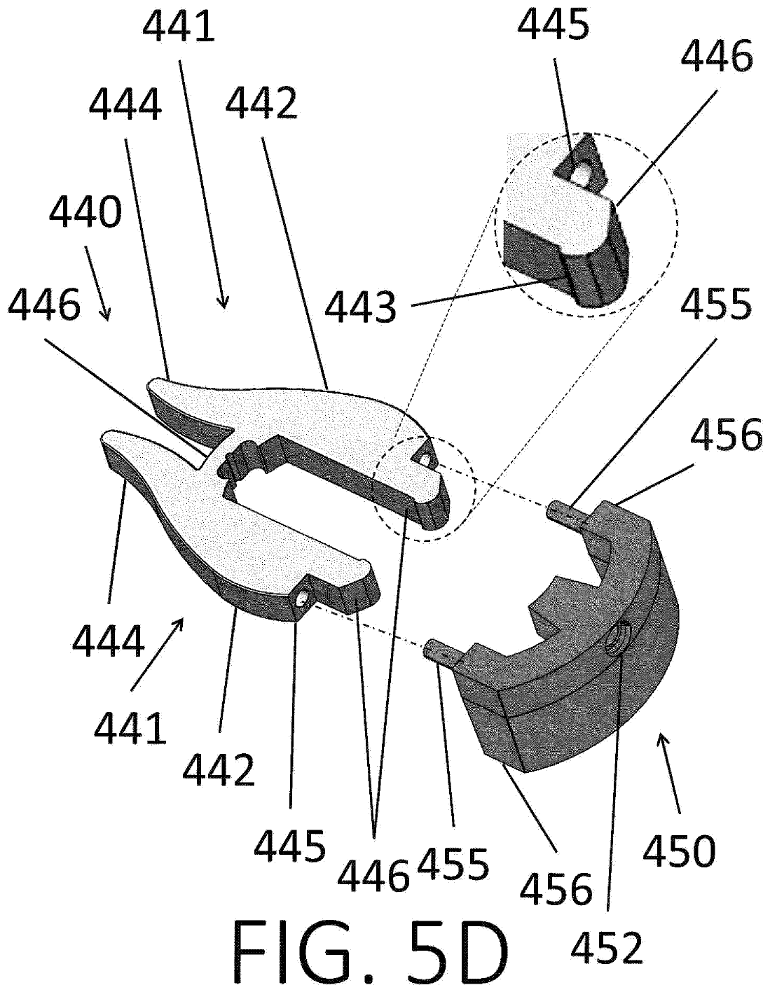

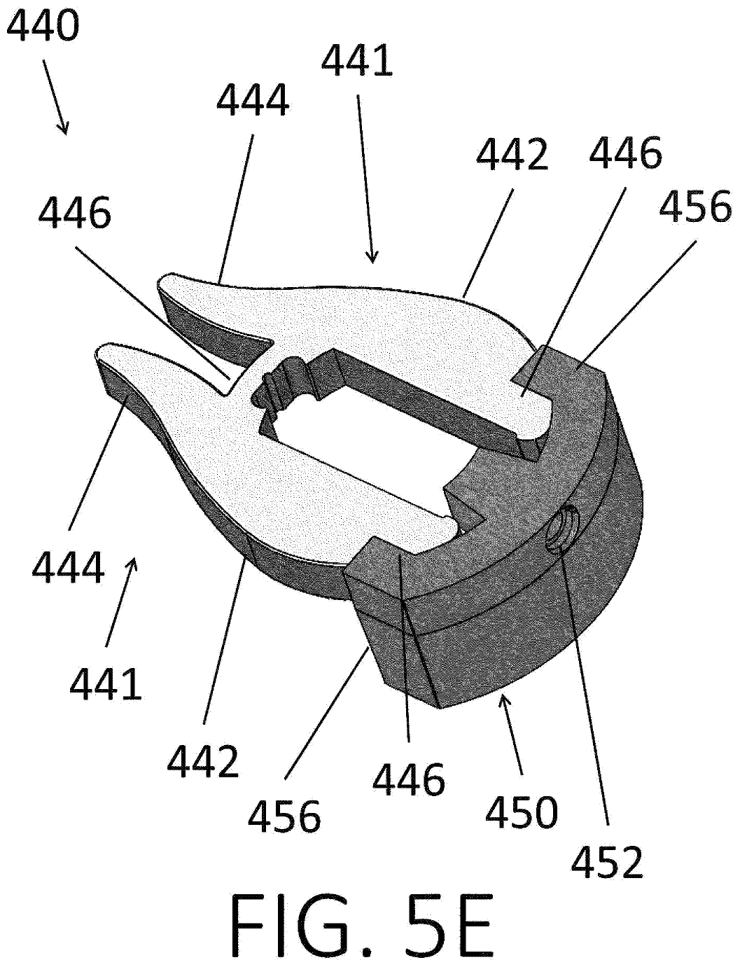

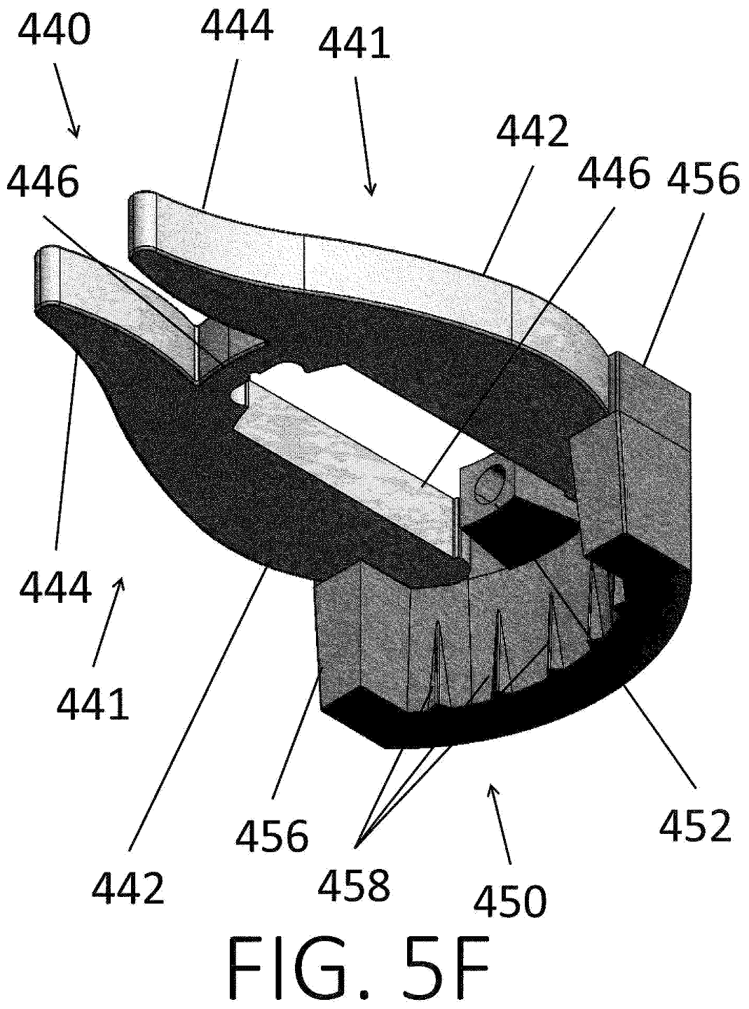

FIGS. 5A-5C illustrate a portion of a trial base 410 that includes a stem 422 and an adaptor 430 at a proximal end of stem 422. Stem 422 is for insertion into the medullary canal of a bone. Stem 422 may optionally include protrusions, such as the blades 428 that extend radially outward therefrom and longitudinally therealong or the protrusions 26, 36 described above, to resist rotation of stem 422 within the medullary canal. Adaptor 420 may be configured to enable connection of a trial humeral head prosthesis or a trial of any other kind of prosthesis to trial base 410. In other arrangements, adaptor 430 may be absent and a trial humeral head prosthesis or a trial of any other kind of prosthesis may be integrally formed with a body located where adaptor 430 is located in the illustrated example.

Adaptor 430 may include a neck 432 located at a distal end of adaptor 430 and thus adjacent to a proximal end of stem 422. Neck 432 may limit the possible depth of insertion of stem 422 into a medullary canal by having a greater radius, measured perpendicularly from longitudinal axis X1, than stem 422 in at least one angular direction. Thus, if trial base 410 is driven into a medullary canal of a prepared bone until a distal surface of neck 432 abuts a proximal surface of the prepared bone, neck 432 will remain accessible from outside the prepared bone. However, neck 432 may optionally also be narrower than more proximal portions of adaptor 430 in at least one direction perpendicular to longitudinal axis X1. As shown in the illustrated example, neck 432 may be narrower than more proximal portions of adaptor 430 when measured perpendicular to both longitudinal axis X1 and lateral axis X2 while being wider than stem 422 when measured perpendicular to longitudinal axis X1 but parallel to lateral axis X2.

As shown in FIG. 5B, a clip 440 may be applied to neck 432. Next, as shown in FIG. 5C, an anchor 450 may be fastened to neck 432 to lock clip 440 to trial base 410 by driving a fastener through an aperture 452 of anchor 450 into a hole 424 in stem 422. Hole 424 is defined in a portion of stem 422 that extends proximally into neck 432 and adaptor 430 and stem 422 are respectively configured so that adaptor 430 may be separated from stem 422 by sliding adaptor 430 along lateral axis X2 relative to stem 422. Locking clip 440 onto neck 432 by fastening anchor 450 to stem 422 as illustrated therefore prevents separation of adaptor 430 from stem 422.

As shown in FIG. 5D, clip 440 includes opposed levers 441 and a hinge 446 that connects the levers 441 to one another. Hinge 446 of the illustrated example is a living hinge that elastically biases levers 441 to respective resting positions, though in other arrangements hinge 446 may be of a type other than a living hinge and may optionally have or lack any features for biasing levers 441.

Each lever 441 includes a tab 444 on one side of hinge 446 and a bar 442 on the other side of hinge 446. Each bar 442, at an end of the bar 442 opposed to tab 444 of the same lever 441, defines a hole 445 and an extension 446. Holes 445 are located so that both extensions 446 are between holes 445. Each extension 446 ends in a hook 443 that extends toward the other extension 446.

Anchor 450 includes pegs 455 sized and spaced to match holes 445. Thus, anchor 450 may be assembled to clip 440 by advancing pegs 455 into holes 445. When anchor 450 is assembled to clip 440 in this way, the spacing of protrusions 446 relative to one another is constrained to that which results from levers 441 being positioned as necessary to align holes 445 with pegs 455. Hooks 443 are too near to one another to permit clip 440 to be removed from neck 432 when clip 440 and anchor 450 are assembled onto neck 432 as shown in FIG. 5C. Clip 440 and anchor 450 are therefore both prevented from being removed from neck 432 by fastener 460.

Anchor 450 also includes walls 456 from which pegs 455 extend. Walls 456 also prevent hooks 443 from moving apart when anchor 450 is assembled to clip 440. In various arrangements, walls 456 may be omitted so the spacing of hooks 443 is limited only by pegs 455 and holes 445 or pegs 455 and holes 445 may be omitted so the spacing of hooks 443 is limited only by walls 456.

Bone may be clamped between a distal extension anchor 450 and stem 422 when anchor 450 is fastened to stem 422 as shown in FIG. 5C to limit rotation of stem 422 relative to the clamped bone. Referring to FIG. 5F specifically, a radially inward side of anchor 450 may optionally include protrusions, such as the blades 458 that extend radially inward therefrom and longitudinally therealong or the protrusions 26, 36 described above, to resist rotation of bone relative to anchor 450 about longitudinal axis X1.

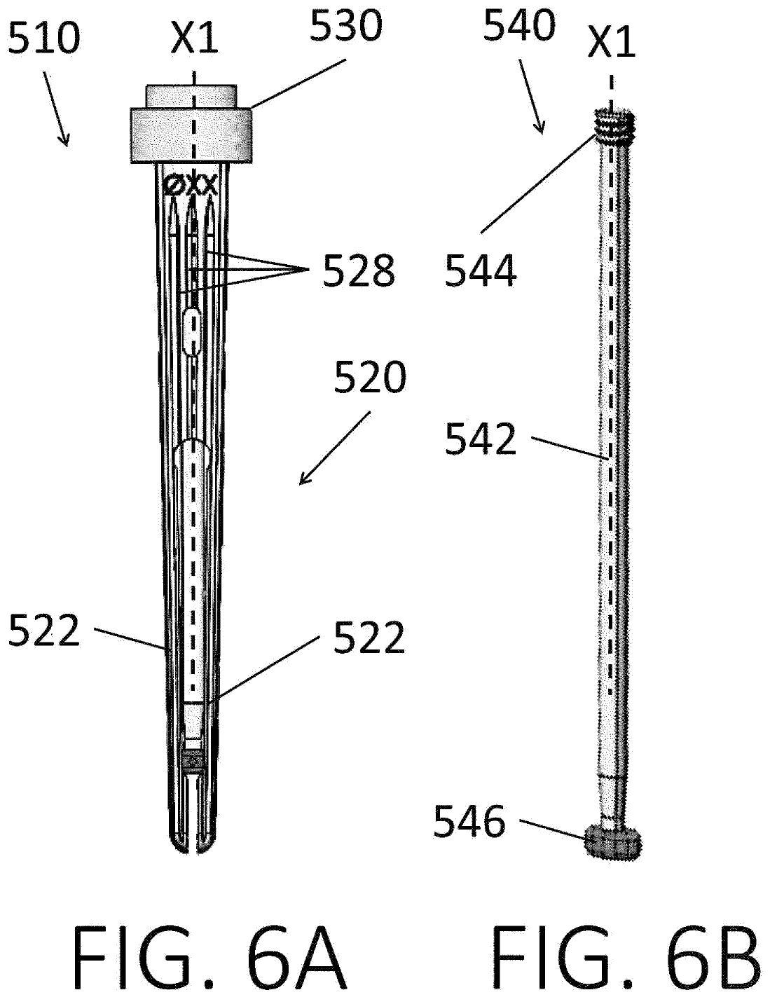

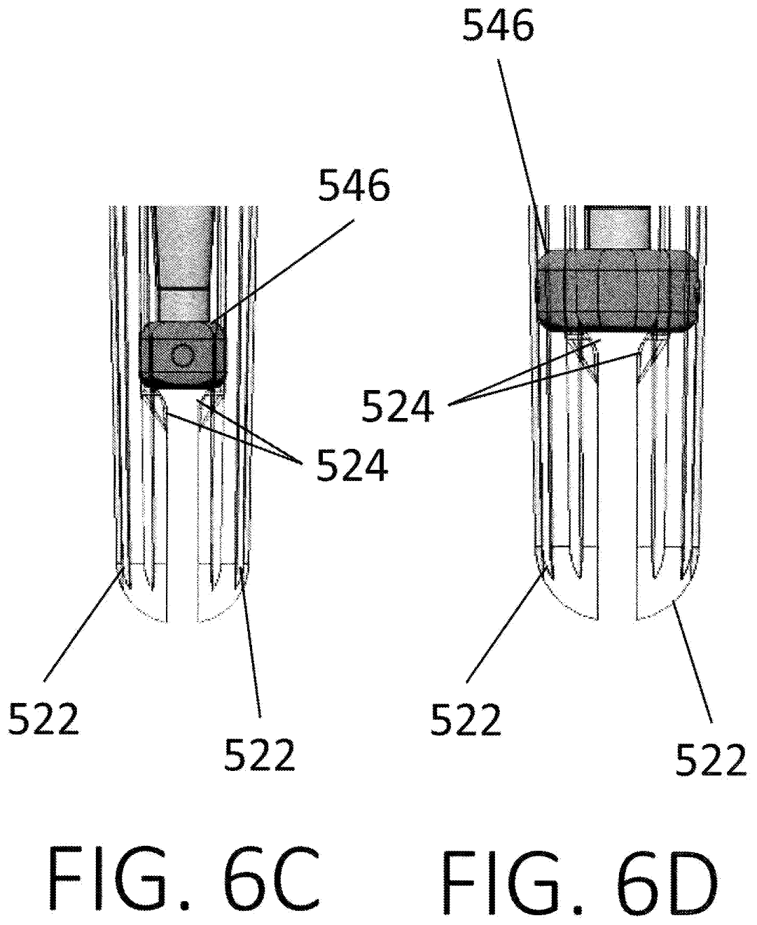

FIG. 6A illustrates a trial base 510 including a stem 520 and a head 530 at a proximal end of stem 520. Stem 520 is for insertion into the medullary canal of a bone, while head 550 may limit the possible depth of insertion by having a greater radius, measured perpendicularly from longitudinal axis X1, than stem 520 in at least one angular direction. In the illustrated example, head 530 has a greater radius than stem 520 across an angular range of 360°, though the angular range may be smaller in other arrangements. Stem 520 may optionally include protrusions, such as blades 528 that extend radially outward therefrom and longitudinally therealong or protrusions 26, 36 described above, to resist rotation of stem 520 within the medullary canal. A distal portion of stem 520 is provided by two opposed fins 522 that are flexibly and resiliently connected to one another at their proximal ends. Though fins 522 are flexibly and resiliently connected to one another in the illustrated arrangement, in other arrangements fins 522 may be hingedly connected at their respective proximal ends.

FIG. 6B shows a control rod 540 that includes a shaft 542, a threaded portion 544 at a proximal end of shaft 542 and a cam 546 at a distal end of shaft 542. Cam 546 has an oval shape from a perspective along longitudinal axis X1. The oval shape has a length and a width, with the length being greater than the width. Threaded portion 544 is threadedly engaged with a threaded aperture at a proximal end of head 530. A proximal end of control rod 540 may be accessible through the threaded aperture so that control rod 540 can be rotated about longitudinal axis X1 by application of a driving tool to the proximal end of control rod 540.

As shown in FIGS. 6C and 6D, cam 546 can be rotated between alternate positions, such as by applying a driving tool to a proximal end of shaft 542, to align either the relatively large length or the relatively small width of cam's 546 oval cross-sectional shape with fins 522. When the width of cam 546 is aligned with fins 522 as shown in FIG. 5C, fins 522 may be relatively close to one another. However, when the length of cam 546 is aligned with fins 522 as shown in FIG. 5D, fins 522 are forced further apart from one another. Trial base 510 may therefore be used by inserting stem 520 into a medullary canal of a bone while the width of cam 546 is aligned with fins 522 as shown in FIG. 5C, then, while stem 520 remains disposed within the medullary canal, driving control rod 540 to rotate about longitudinal axis X1 relative to stem 520 so that the length of cam 546 becomes aligned with fins 522 as shown in FIG. 5D. Forcing fins 522 apart by rotating cam 546 may cause fins 522 to engage the bone from within, thereby inhibiting rotation of trial base 510 about longitudinal axis X1 relative to the bone.

Opposed, radially inner faces of fins 522 may optionally include ramps 524 that are inclined to be relatively far from longitudinal axis X1 at their proximal ends and relatively near to longitudinal axis X1 at their distal ends. Thus, if trial base 510 is used in a bone with a particularly large internal diameter, control rod 540 may be rotated to threadedly advance distally until cam 546 contacts ramps 524 at sufficiently distal locations to push fins 522 into engagement with the bone.

Though only two fins 522 are shown in the illustrated example, the distal end of stem 520 in other arrangements may be provided by any plural number of fins 522. In such other arrangements, cam 546 can have any shape suitable to alternately allow fins 522 to remain close together or force fins 522 apart as cam 546 is rotated about longitudinal axis X1. For example, in arrangements with three fins 522, cam 546 may have a triangular shape, and so on.

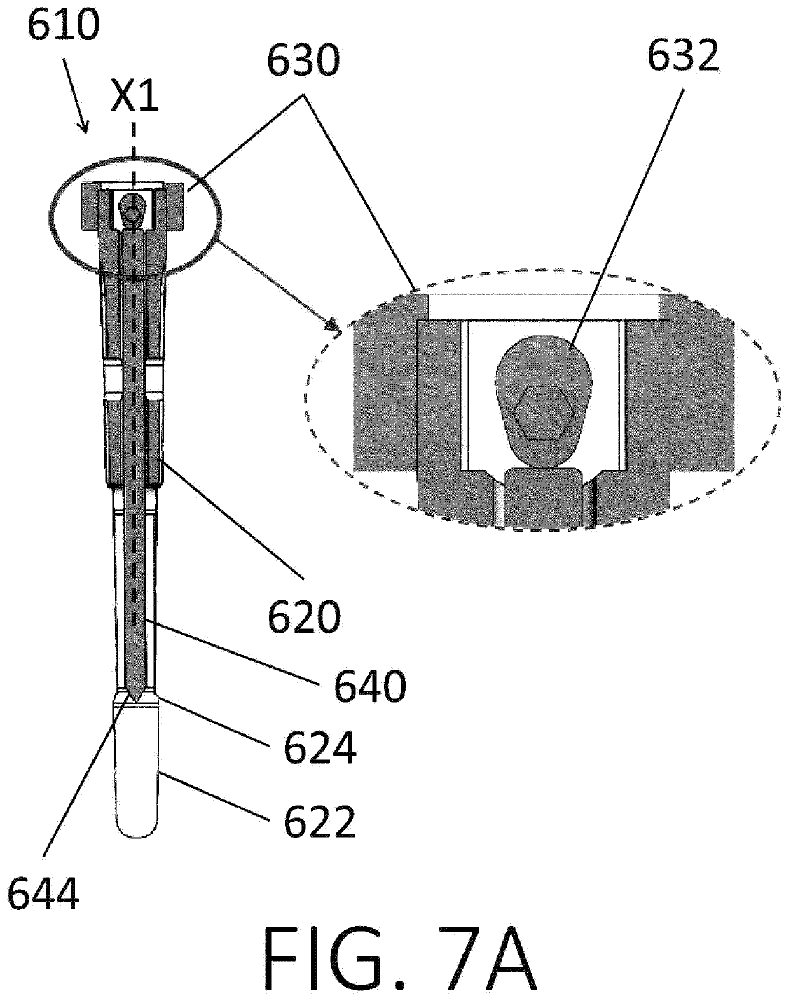

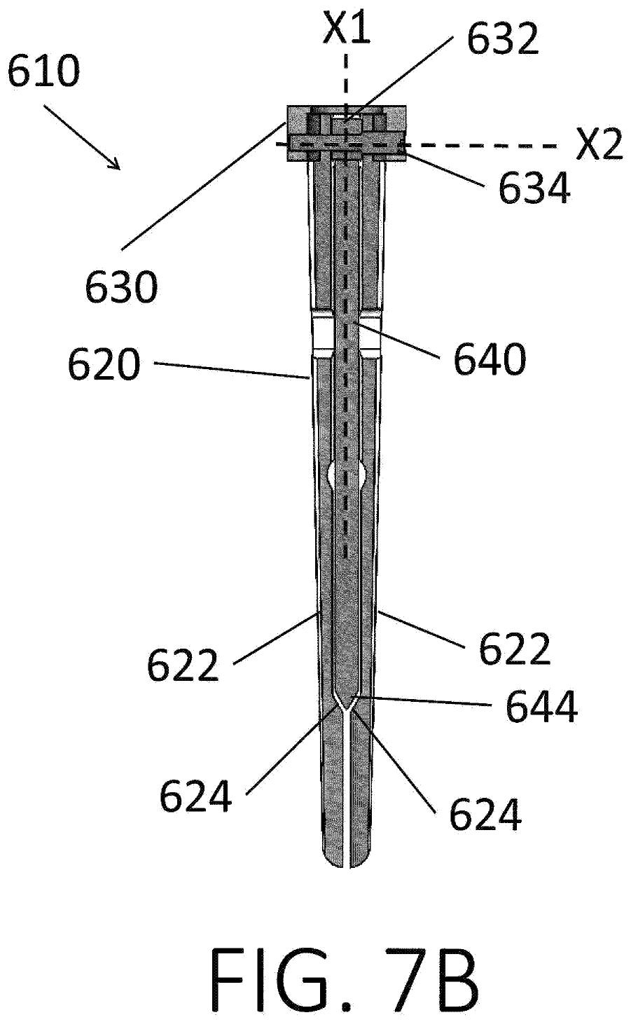

FIGS. 7A and 7B illustrate a trial base 610 including a stem 620 and a head 630 at a proximal end of stem 620. Stem 620 is for insertion into the medullary canal of a bone, while head 650 may limit the possible depth of insertion by having a greater radius, measured perpendicularly from longitudinal axis X1, than stem 620 in at least one angular direction. In the illustrated example, head 630 has a greater radius than stem 620 across an angular range of 360°, though the angular range may be smaller in other arrangements. Stem 620 may optionally include protrusions, such as blades that extend radially outward therefrom and longitudinally therealong or protrusions 26, 36 described above, to resist rotation of stem 620 within the medullary canal. A distal portion of stem 620 is provided by two opposed fins 622 that are flexibly and resiliently connected to one another at their proximal ends. Though fins 622 are flexibly and resiliently connected to one another in the illustrated arrangement, in other arrangements fins 622 may be hingedly connected at their respective proximal ends. Though only two fins 722 are shown in the illustrated example, the distal end of stem 720 in other arrangements may be provided by any plural number of fins 722.

A control rod 640 extends within stem 620 along longitudinal axis A1. Head 630 contains a cam 632 and an axle 634 on which cam 632 is rotationally fixed. Thus, turning axle 634 also turns cam 632. To facilitate rotation of axle 634, an end of axle 634 may be accessible through an opening in head 630, and that end of axle 634 may include an adaptor in the form of any convex or concave feature enabling transmission of torque about lateral axis X2 to axle 634. Axle 634 extends along, and is rotatable about, lateral axis X2, so driving axle 634 causes cam 632 to rotate about lateral axis X2.

Cam 632 is asymmetrical about lateral axis X2, so a longitudinal position of a distalmost point of cam 632 will vary as cam 632 rotates about lateral axis X2. Control rod 640 is retained within trial base 610 so that a proximal end of control rod 640 is adjacent cam 632 and the position of control rod 640 along longitudinal axis X1 depends on an angular position of cam 632 about lateral axis X2. In the illustrated example, the asymmetry of cam 632 results both from cam 632 having a non-circular, and specifically egg-shaped, cross-section on a plane normal to lateral axis X2 and axle 634 extending through cam 632 at a point offset from the center of that egg shaped cross-section. However, in other examples, cam 632 may have a circular cross-section having a center point offset from axle 634 or cam 632 may have any non-circular cross-section that is centered on axle 634.

Opposed, radially inner faces of fins 622 include ramps 624 that are inclined to be relatively far from longitudinal axis X1 at their proximal ends and relatively near to longitudinal axis X1 at their distal ends. A distal end 644 of control rod 640 is positioned to contact ramps 624 at different locations along longitudinal axis X1 depending on the angular position of cam 632 about lateral axis X2. Thus, control rod 640 will force fins 622 apart from one another and radially away from longitudinal axis X2 as axle 634 is rotated to move the longitudinal location of the distalmost point of cam 632 distally. Trial base 610 may therefore be used by inserting stem 620 into a medullary canal of a bone while cam 632 is in an angular position about lateral axis X2 that allows control rod 640 to occupy a relatively proximal location within trial base 610, then, while stem 620 remains disposed in the medullary canal, rotating axle 634 to turn cam 632 to push rod 640 distally and force fins 622 apart until fins 622 engage the bone from within. Engaging the bone from within in this manner inhibits rotation of trial base 610 about longitudinal axis X1 within the bone. Distal end 644 may optionally be wedge shaped, such as in the illustrated example, though non-wedge shaped distal ends 644 according to other arrangements are be suitable for usage with ramps 624.

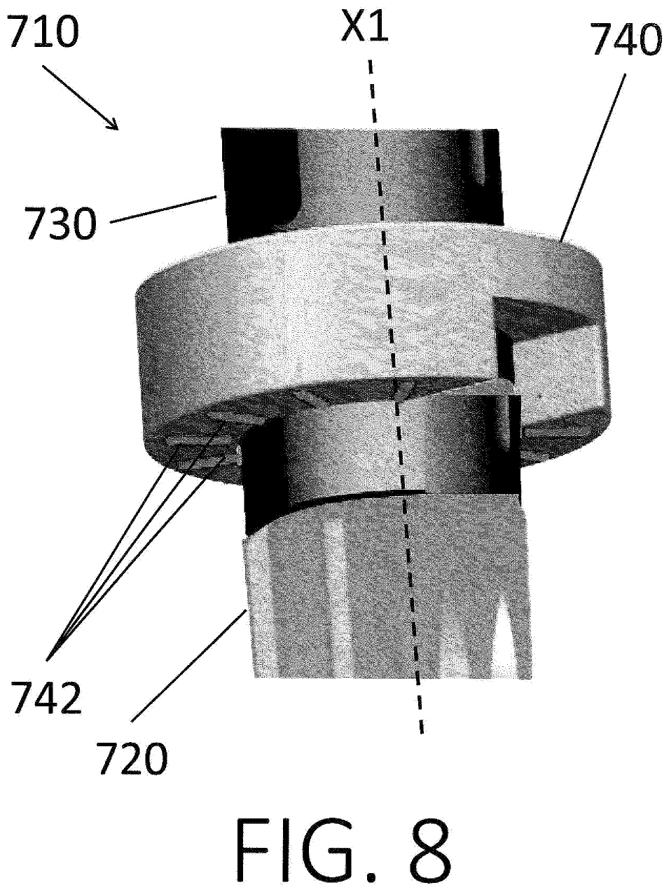

FIG. 8 illustrates a portion of a trial base 710 that includes a stem 720 and a collar 740 at a proximal end of stem 322. Stem 720 is for insertion into the medullary canal of a bone, while collar 740 may limit the possible depth of insertion by having a greater radius, measured perpendicularly from longitudinal axis X1, than stem 720 in at least one angular direction. In the illustrated example, head 740 has a greater radius than stem 720 across an angular range of 360°, though the angular range may be smaller in other arrangements. Stem 720 may optionally include protrusions, such as blades that extend radially outward therefrom and longitudinally therealong or the protrusions 26, 36 described above, to resist rotation of stem 720 within the medullary canal. Trial base 710 may also include an adaptor 730 located proximally of stem 720. Adaptor 730 may be configured to enable connection of a trial humeral head prosthesis or a trial of any other kind of prosthesis to trial base 710. In other arrangements, adaptor 730 may be absent and a trial humeral head prosthesis or a trial of any other kind of prosthesis may be integrally formed with a body located where adaptor 730 is located in the illustrated example. Collar 740 may optionally be either permanently fixed to adaptor 730 or selectably fixable to adaptor 730 so that collar 740 may be longitudinally slidable along adaptor 730 when unfixed.

Several protrusions 742 are defined on a distal surface of collar 740. Protrusions 742 extend distally from collar 740, and will therefore engage a proximal surface of the bone having the medullary canal in which stem 720 is inserted. In the illustrated example, protrusions 742 are blades that extend radially away from longitudinal axis X2. However, in other examples, protrusions 742 may be any distally extending features that cooperate with distal forces upon trial base 710 to engage the bone and inhibit rotation of trial base 710 about longitudinal axis X1 relative to the bone. Collar 740 and protrusions 742 therefore inhibit rotation of trial base 710 about longitudinal axis X1 relative to the bone when stem 720 is fully inserted into the bone without the need for any additional steps to create engagement between trial base 710 and the bone.

Any of the devices described in the present disclosure can be made of any sufficiently strong, durable, and biocompatible materials. Examples of suitable metals or metal alloys suitable for this purpose include stainless steel, titanium, nitinol, and any other biocompatible metals or metal alloys. Examples of suitable polymers include high-density polyethylene (“HDP”), polymethylmethacrylate (“PMMA”), polyetheretherketone (“PEEK”), or any other rigid and biocompatible polymer. In further examples, some or all components of any of the foregoing devices may be constructed of ceramic.

Although the concepts herein have been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A prosthetic trial base, comprising:
a hinge;
two levers connected to one another by the hinge, each lever comprising a tab extending proximally of the hinge and a fin extending distally of the hinge;
a head movable between the tabs to force the tabs apart from one another.

2. The prosthetic trial base of claim 1, wherein proximal and distal are opposite directions along a longitudinal axis, and the head is drivable along the longitudinal axis.

3. The prosthetic trial base of claim 1, wherein the levers are constructed to create a direct correlation between an angle between the tabs and an angle between the fins.

4. The prosthetic trial base claim 1, wherein each of the tabs has a ramped surface facing the other tab.

5. The prosthetic trial base of claim 1, wherein the head is cone or dome shaped.

6. The prosthetic trial base of claim 1, wherein the head is drivable relative to a lid that is connected to the levers.

7. The prosthetic trial base of claim 6, comprising a collar connecting the lid to the levers and defining a bore in which the tabs are received.

8. The prosthetic trial base of claim 7, comprising a bolt that includes the head and is threadedly engaged with the lid.

9. The prosthetic trial base of claim 8, wherein proximal and distal are opposite directions along a longitudinal axis, and the lid is rotationally fixed relative to the collar about the longitudinal axis.

10. The prosthetic trial base of claim 7, wherein the fins extend distally of the collar.

11. A prosthetic trial base, comprising:
a collar;
two fins extending distally from the collar and each being connected to a hinge;
a head drivable relative to the collar to force each fin to rotate relative to the hinge.

12. The prosthetic trial base of claim 11, wherein the collar defines a bore within which the head is received.

13. The prosthetic trial base of claim 12, comprising two levers, wherein each lever includes a respective one of the fins and a tab that extends into the collar.

14. The prosthetic trial base of claim 13, wherein the levers are hingedly connected to one another.

15. The prosthetic trial base of claim 11, wherein proximal and distal are opposite directions along a longitudinal axis and the each fin is connected to the hinge at a longitudinally fixed point.

16. A method of using a prosthetic trial base comprising:
inserting two hingedly connected fins of the trial base into a medullary canal of a bone; and
driving a head of the trial base relative to the fins to cause the fins to pivot relative to one another and into engagement with the bone.

17. The method of claim 16, wherein the trial base comprises two levers, each of the levers comprises a respective one of the fins and a tab, and driving the head comprises advancing the head between the tabs to force the tabs apart.

18. The method of claim 17, wherein the levers are connected to one another by a hinge and driving the head comprises advancing the head toward the hinge.

19. The method of claim 16, wherein the fins extend distally from a collar and inserting the two fins into the medullary canal comprises advancing the fins into the medullary canal until a distal surface of the collar contacts a proximal surface of the bone.

20. The method of claim 16, comprising attaching and removing multiple different trial articular prostheses to the trial base in series while the fins remain in engagement with the bone.
